(19) United States
(12) Patent Application Publication
(10) Pub. No.: US 2022/0401447 A1
(43) Pub. Date: Dec. 22, 2022

(54) TOPICAL DELIVERY OF TOFACITINIB USING IONIC LIQUID

(71) Applicant: CAGE BIO, INC., San Carlos, CA (US)

(72) Inventors: Nitin JOSHI, San Carlos, CA (US); Kevin GELSTON, San Carlos, CA (US); Marina SHEVACHMAN, San Carlos, CA (US)

(21) Appl. No.: 17/775,269

(22) PCT Filed: Nov. 7, 2020
(86) PCT No.: PCT/US2020/059580

§ 371 Date: May 6, 2022

Related U.S. Application Data
(60) Provisional application No. 62/933,300, filed on Nov. 8, 2019.

(51) Int. Cl. A61K 31/519; A61K 9/00; A61K 47/12; A61K 47/18; A61K 47/10; A61K 9/06

(52) U.S. Cl. CPC A61K 31/519; A61K 9/0014; A61K 47/12; A61K 47/186; A61K 47/10; A61K 9/06

(57) ABSTRACT

One aspect of the present disclosure is directed to a method of delivering tofacitinib to or through a skin. Another aspect of the present disclosure is directed to a method of locally inhibiting activity of at least one of JAK-1, JAK-2, TYK-2, and JAK-3 within an epidermis layer, a dermis layer, a subcutaneous tissue layer, or a muscle tissue. Another aspect of the present disclosure is directed to a topical composition, comprising tofacitinib or a pharmaceutically acceptable salt thereof, and an ionic liquid in an amount sufficient to allow a therapeutically effective amount of tofacitinib to reach a target depth within or beyond the skin.

### BACKGROUND OF THE DISCLOSURE

Tofacitinib, a janus kinase (JAK) inhibitor, is sometime orally administered to treat rheumatoid arthritis, psoriatic arthritis and ulcerative colitis.

### SUMMARY OF THE DISCLOSURE

One aspect of the present disclosure is directed to a method of delivering tofacitinib to or through a skin. Another aspect of the present disclosure is directed to a method of locally inhibiting activity of at least one of JAK-1, JAK-2, TYK-2, and JAK-3 within an epidermis layer, a dermis layer, a subcutaneous tissue layer, or a muscle tissue. Another aspect of the present disclosure is directed to a topical composition, comprising tofacitinib or a pharmaceutically acceptable salt thereof, and an ionic liquid in an amount sufficient to allow a therapeutically effective amount of tofacitinib to reach a target depth within or beyond the skin.

Disclosed herein, in certain embodiments, is a method of delivering tofacitinib to or through a skin, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

Disclosed herein, in certain embodiments, is a method of locally inhibiting activity of at least one of JAK-1, JAK-2, TYK-2, and JAK-3 within an epidermis layer, a dermis layer, a subcutaneous tissue layer, or a muscle tissue, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

Disclosed herein, in certain embodiments, is a topical composition, comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows delivery of two non-limiting topical compositions comprising tofacitinib and ionic liquid according to some embodiments of the present disclosure, in comparison with a first control composition by replacing the ionic liquid with a same amount of 2-(2-ethoxyethoxy)ethanol, and a second control composition comprising tofacitinib, a PEG-based ointment containing 2% oleyl alcohol.

### DETAILED DESCRIPTION OF THE DISCLOSURE

Disclosed herein, in certain embodiments, is a topical composition, comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

### Tofacitinib

3-((3R, 4R)-4-Methyl-3-[methyl-(7H-pyrrolo[2,3-d]pyrimidin-4-yl)-amino]-piperidin-1-yl)-3-oxopropionitrile, i.e. tofacitinib, has the following structural formula

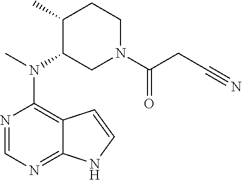 [SMILES: C[C@@H]1CCN(C(=O)CC#N)C[C@@H]1N(C)c1ncnc2[nH]ccc12]

The synthesis of 3-((3R, 4R)-4-methyl-3-[methyl-(7H-pyrrolo[2, 3-d]-pyrimidin-4-yl)-amino]-piperidin-1-yl)-3-oxopropionitrile is described in WO 2001/42246 and WO 2002/096909, commonly assigned to the assignee of the present invention and which are incorporated herein by reference in their entirety. The preparation of 3-((3R, 4R)-4-methyl-3-[methyl-(7H-pyrrolo[2, 3-d]-pyrimidin-4-yl)-amino]-piperidin-1-yl)-3-oxopropionitrile mono citrate salt is described in U.S. Pat. No. 6,965,027.

Tofacitinib and its pharmaceutically acceptable salt such as the mono citrate salt are also useful as inhibitors of protein kinases, such as the enzyme Janus Kinase (JAK). As such, tofacitinib and its pharmaceutically acceptable salt such as the mono citrate salt are sometimes orally administered therapy as immunosuppressive agents for organ transplants, xeno transplantation, lupus, multiple sclerosis, rheumatoid arthritis, psoriasis, Type I diabetes and complications from diabetes, cancer, asthma, atopic dermatitis, autoimmune thyroid disorders, ulcerative colitis, Crohn's disease, Alzheimer's disease, Leukemia and other indications where immunosuppression would be desirable. The present disclosure relates to administration of tofacitinib topically into or through the skin, which present unique challenges especially when the underlying diseases or conditions require delivery of therapeutically effective amount of tofacitinib at various target depths into or through the skin.

### Ionic Liquids

Described herein, in certain embodiments, are compositions comprising an ionic liquid comprising a choline cation and a fatty acid anion. In some embodiments, the composition further comprises a pharmaceutically acceptable solvent. In some embodiments, the fatty acid is myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is geranic acid. In some embodiments, the fatty acid comprises 9 to 14 carbons. In some embodiments, the ionic liquid is liquid at room temperature. In some embodiments, the ionic liquid is liquid below 100° C.

In some embodiments, the ionic liquid is a deep eutectic solvent (DES). In some embodiments, a DES comprises excess carboxylate which precludes 1:1 ion pairing. In some embodiments, a DES further comprises a hydrogen-bond donor. In some embodiments, the hydrogen-bond donor is urea or citric acid. In some embodiments, the solvent properties of a DES are adjusted by changing the hydrogen-bond donor. In some embodiments, the ammonium salt of a DES interacts with a hydrogen-bond donor. In some embodiments, the DES has a melting point lower than either of the individual components (e.g. fatty acid and choline).

In some embodiments, the ionic liquid comprises a molar ratio of a choline cation to a fatty acid anion of 1:0.5 to 1:10. In some embodiments, the molar ratio of the choline cation to the fatty acid anion is about 1:0.5, 1:0.6, 1:0.7, 1:0.8, 1:0.9, 1:1.0; 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9. 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or about 1:10. In some embodiments, the molar ratio of the choline cation to the fatty acid anion is about 1:1.1, 1:1.2, 1:1.3, 1:1.4, 1:1.5, 1:1.6, 1:1.7, 1:1.8, 1:1.9, or 1:2.0.

In some embodiments, the choline cation and fatty acid anion are in a molar ratio in the ionic liquid. In some embodiments, the choline cation and fatty acid anion are in a molar ratio of 1:1. In some embodiments, the term Composition B is used herein to refer to a composition or an ionic liquid comprising a 1:1 molar ratio of choline cation to geranic acid anion. In some embodiments, Composition B does not comprise water.

In other embodiments, the choline cation and fatty acid anion are in a molar ratio of 1:2. In some embodiments, the term Composition A is used herein to refer to a composition or an ionic liquid comprising a 1:2 molar ratio of choline cation to geranic acid anion. In some embodiments, Composition A does not comprise water.

In some embodiments, the chemical structure of choline is:

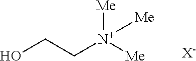

wherein X⁻ is a pharmaceutically acceptable anion.

In some embodiments, term choline refers to the class of quaternary ammonium salts containing the N,N,N-trimethylethanolammonium cation. In some embodiments, the X⁻ on the right of the structure of choline denotes a pharmaceutically acceptable anion. In some embodiments the X⁻ is bicarbonate, carbonate, acetate, citrate, tartarate, bitartarate, lactate, chloride, bromide, or iodide. In some embodiments, the X⁻ is bicarbonate. In some embodiments, the choline is an anti-inflammatory agent.

In some embodiments, choline is in the form of a pharmaceutically acceptable salt. The type of pharmaceutical acceptable salts, include, but are not limited to acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the chemical structure of geranic acid, or 3,7-dimethyl-2,6-octadienoic acid, is:

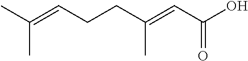

In some embodiments, geranic acid is in the form of a pharmaceutically acceptable salt. The type of pharmaceutical acceptable salts, include, but are not limited to salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Examples of acceptable organic bases include, but are not limited to, ethanolamine, diethanolamine, triethanolamine, tromethamine, and N-methylglucamine. Examples of acceptable inorganic bases include, but are not limited to, aluminum hydroxide, calcium hydroxide, potassium hydroxide, sodium carbonate, and sodium hydroxide.

In some embodiments, the choline and the fatty acid are synthesized using any suitable standard synthetic reactions. In some embodiments, the reactions are employed in a linear sequence to provide the compounds or they may be used to synthesize fragments which are subsequently joined by any suitable method. In some embodiments, the starting material used for the synthesis of choline or fatty acid is synthesized or are obtained from commercial sources. In some embodiments, geranic acid is purified from the commercially available technical grade (Sigma-Aldrich, St. Louis, Mo.) by repeated (5-7×) recrystallization from a solution of 70 wt % geranic acid/30 wt % acetone at −70° C. In some embodiments, purity of the geranic acid is assessed by ¹H NMR spectroscopy and conductivity measurements. In some embodiments, the term geranic acid refers to a geranic acid or a salt thereof. In some embodiments, the geranic acid is an anti-microbial agent.

Tofacitinib Topical Composition

In some embodiments, each component in a composition, such as the tofacitinib, ionic liquid, the pharmaceutically acceptable carrier, and optionally other components, is described a percent (%) of the composition. In some embodiments, the % of the composition is a percent concentration volume/volume (v/v) or a percent concentration weight/volume (w/v).

Disclosed herein, in certain embodiments, is a topical composition, comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

In some embodiments, the pharmaceutical composition comprises about 0.01% to about 10% of tofacitinib or a pharmaceutically acceptable salt thereof. In some embodiments, the pharmaceutical composition comprises about 0.1% to about 8% of tofacitinib or a pharmaceutically acceptable salt thereof. In some embodiments, the topical ointment comprises about 0.1% to about 5% of tofacitinib or a pharmaceutically acceptable salt thereof. In some embodiments, the target depth is from about 0.05 mm to about 20 mm. In some embodiments, the target depth is from about 0.1 mm to about 15 mm. In some embodiments, the target depth is from about 1 mm to about 10 mm

In some embodiments, the pharmaceutical composition allows therapeutically effective amount of tofacitinib to reach at least an epidermis layer. In some embodiments, the pharmaceutical composition allows therapeutically effective amount of tofacitinib to reach at least a dermis layer.

In some embodiments, the pharmaceutical composition allows therapeutically effective amount of tofacitinib to reach at least a subcutaneous tissue layer. In some embodiments, the pharmaceutical composition allows therapeutically effective amount of tofacitinib to reach at least a muscle tissue.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is selected from the group consisting oleic acid, geranic acid, hexanoic acid, and malonic acid. In some embodiments, the fatty acid is geranic acid.

In some embodiments, the ionic liquid is a deep eutectic solvent (DES). In some embodiments, the ionic liquid comprises the choline cation and fatty acid anion in a molar ratio in a range of 1:1 to 1:4 of choline cation to fatty acid anion. In some embodiments, the ionic liquid comprises the choline cation and fatty acid anion in a molar ratio of 1:2 of choline cation to fatty acid anion.

In some embodiments, the pharmaceutical composition consists essentially of tofacitinib and the ionic liquid. In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an ointment base.

In some embodiments, the ionic liquid is present in the pharmaceutical composition at a concentration of from about 1% to about 99%. In some embodiments, the ionic liquid is present in the pharmaceutical composition at a concentration of from about 5% to about 90%. In some embodiments, the ionic liquid is present in the pharmaceutical composition at a concentration of from about 10% to about 80%. In some embodiments, the ionic liquid is present in the pharmaceutical composition at a concentration of from about 15% to about 70%. In some embodiments, the ionic liquid is present in the pharmaceutical composition at a concentration of from about 20% to about 60%. In some embodiments, the ionic liquid is present in the pharmaceutical composition at a concentration of from about 30% to about 50%. In some embodiments, the ionic liquid is present in the pharmaceutical composition at a concentration of about 35% to about 45%. In some embodiments, the ionic liquid is present in the pharmaceutical composition at a concentration of about 40%.

In some embodiments, the pharmaceutical composition further comprises a penetration enhancer. In some embodiments, the penetration enhancer is 2-(2-ethoxyethoxy)ethanol or oleyl alcohol. In some embodiments, the penetration enhancer is 2-(2-ethoxyethoxy)ethanol. In some embodiments, the pharmaceutical composition comprises from about 1% to about 20% of 2-(2-ethoxyethoxy)ethanol. In some embodiments, the pharmaceutical composition comprises from about 5% to about 15% 2-(2-ethoxyethoxy)ethanol. In some embodiments, the pharmaceutical composition comprises from about 10% 2-(2-ethoxyethoxy)ethanol.

In some embodiments, the pharmaceutical composition is essentially free of 2-(2-ethoxyethoxy)ethanol. In some embodiments, the pharmaceutical composition is essentially free of oleyl alcohol.

In some embodiments, the composition comprises the ionic liquid in a concentration of about 0.1% to 99%. In some embodiments, the composition comprises the ionic liquid in a concentration of about 1% to 40%. In some embodiments, the composition comprises the ionic liquid in a concentration of about 1% to 20%. In some embodiments, the composition comprises the ionic liquid in a concentration of about 5% to 20%. In some embodiments, the composition comprises the ionic liquid in a concentration of about 5% to 40%. In some embodiments, the composition comprises the ionic liquid in a concentration of about 20% to 40%. In some embodiments, the composition comprises the ionic liquid in a concentration of about 20% to 60%. In some embodiments, the composition comprises the ionic liquid in a concentration of about 20% to 80%.

In some embodiments, the composition comprises the ionic liquid in a concentration of about 0.1% to 99%, and the pharmaceutically acceptable solvent in a concentration of about 1% to about 99.9%. In some embodiments, the composition comprises the ionic liquid in a concentration of about 1% to 40%, and the pharmaceutically acceptable solvent in a concentration of about 60% to about 99%. In some embodiments, the composition comprises the ionic liquid in a concentration of about 20% to 40%, and the pharmaceutically acceptable solvent in a concentration of about 80% to about 99%. In some embodiments, the composition comprises the ionic liquid in a concentration of about 20% and the pharmaceutically acceptable solvent in a concentration of about 80%. In some embodiments, the composition comprises the ionic liquid in a concentration of about 40% and the pharmaceutically acceptable solvent in a concentration of about 60%.

In some embodiments, the composition comprises the ionic liquid in a concentration of about 1% to 50%, and the pharmaceutically acceptable solvent in a concentration of about 50% to 99%. In some embodiments, the composition comprises the ionic liquid in a concentration of about 1% to 50%, and water in a concentration of about 50% to 99%. In some embodiments, the water is deionized water or water.

In some embodiments, the composition comprises the ionic liquid in a concentration of about 1% to 50%, a pharmaceutically acceptable solvent in a concentration of about 1% to 50%, and a gelling agent in a concentration of about 1 to 5%. In some embodiments, the composition comprises the ionic liquid in a concentration of about 1% to 50%, and water in a concentration of about 1% to 50%.

In some embodiments, the pharmaceutically acceptable solvent is diisopropyl adipate. In some embodiments, the composition comprises diisopropyl adipate in a concentration of about 20%. In some embodiments, the composition comprises the ionic liquid in a concentration of about 1% to 40%, and diisopropyl adipate in a concentration of about 60% to about 99%.

In some embodiments, the composition comprises a gel base in a concentration of about 50% to 90% of the composition. In some embodiments, the composition comprises a gel base in a concentration of about 50%, 60%, 70%, 80%, or 90% of the composition.

In some embodiments, preparing an ionic liquid comprising a choline cation and a fatty acid anion comprises: (a) mixing choline and a fatty acid in a solvent at room temperature in a predetermined ratio; and (b) removing the solvent in vacuo. In some embodiments, the fatty acid is geranic acid. In some embodiments, the solvent is water. In a particular embodiment, the water is deionized water. In some embodiments, removing the solvent comprises rotary evaporation. In some embodiments, removing the solvent comprises heating the ionic liquid, applying a vacuum to the ionic liquid, or a combination thereof. In some embodiments, preparing the ionic liquid further comprises drying the ionic liquid. In some embodiments, heating the ionic liquid comprises heating the ionic liquid to 60° C. In some embodiments, the heating is done for at least 10 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours. In some embodiments, the vacuum is applied at −100 kPa. In some embodiments, the vacuum is applied for at least 10 minutes, 20 minutes, 30 minutes, 1 hours, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours.

In some embodiments, the ionic liquid has had the solvent used in the ionic liquid preparation process removed. In some embodiments, the ionic liquid does not comprise water.

In some embodiments, choline is choline bicarbonate. In some embodiments, the choline is choline in an 80% wt solution of choline bicarbonate. In some embodiment, the predetermined ratio is a ratio of 1:1, 1:2, 1:3, or 1:4 of a choline cation: fatty acid anion. In one embodiment, the ratio is a molar ratio. In another embodiment, the ratio is ratio by weight.

In some embodiments, isolating the composition further comprises purifying the ionic liquid. In some embodiments, purifying the ionic liquid comprises using conventional techniques, including, but not limited to, filtration, distillation, crystallization, and chromatography. In some embodiments, preparing the ionic liquid further comprises isolating the purified ionic liquid

### Tofacitinib Topical Delivery

Disclosed herein, in certain embodiments, is a method of delivering tofacitinib to or through a skin, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

In some embodiments, the pharmaceutical composition comprises about 0.01% to about 10% of tofacitinib or a pharmaceutically acceptable salt thereof. In some embodiments, the pharmaceutical composition comprises about 0.1% to about 8% of tofacitinib or a pharmaceutically acceptable salt thereof. In some embodiments, the pharmaceutical composition comprises about 0.1% to about 5% of tofacitinib or a pharmaceutically acceptable salt thereof.

In some embodiments, the target depth is from about 0.05 mm to about 20 mm. In some embodiments, the target depth is from about 0.1 mm to about 15 mm. In some embodiments, the target depth is from about 1 mm to about 10 mm.

In some embodiments, the pharmaceutical composition allows therapeutically effective amount of tofacitinib to reach at least an epidermis layer. In some embodiments, the amount of tofacitinib at the epidermis layer is sufficient to inhibit activity of at least one of JAK-1, JAK-2, TYK-2, and JAK-3 within the epidermis layer.

In some embodiments, the pharmaceutical composition allows therapeutically effective amount of tofacitinib to reach at least a dermis layer. In some embodiments, the amount of tofacitinib at the dermis layer is sufficient to inhibit activity of at least one of JAK-1, JAK-2, TYK-2, and JAK-3 within the dermis layer.

In some embodiments, the pharmaceutical composition allows therapeutically effective amount of tofacitinib to reach at least a subcutaneous tissue layer. In some embodiments, the amount of tofacitinib at the subcutaneous tissue layer is sufficient to inhibit activity of at least one of JAK-1, JAK-2, TYK-2, and JAK-3 within the subcutaneous tissue layer.

In some embodiments, the pharmaceutical composition allows therapeutically effective amount of tofacitinib to reach at least a muscle tissue. In some embodiments, the amount of tofacitinib at the muscle tissue is sufficient to inhibit activity of at least one of JAK-1, JAK-2, TYK-2, and JAK-3 within the muscle tissue.

In some embodiments, the total amount of tofacitinib reaching at least the epidermis layer provided by the pharmaceutical composition is at least 1.5 times the total amount of tofacitinib reaching at least the epidermis layer provided by a control composition replacing the ionic liquid with same amount of 2-(2-ethoxyethoxy)ethanol. In some embodiments, the total amount of tofacitinib reaching at least the epidermis layer provided by the pharmaceutical composition is at least 2 times the total amount of tofacitinib reaching at least the epidermis layer provided by a control composition replacing the ionic liquid with same amount of 2-(2-ethoxyethoxy)ethanol. In some embodiments, the total amount of tofacitinib reaching at least the epidermis layer provided by the pharmaceutical composition is at least 2.5 times the total amount of tofacitinib reaching at least the epidermis layer provided by a control composition replacing the ionic liquid with same amount of 2-(2-ethoxyethoxy)ethanol. In some embodiments, the total amount of tofacitinib reaching at least the epidermis layer provided by the pharmaceutical composition is at least 3 times the total amount of tofacitinib reaching at least the epidermis layer provided by a control composition replacing the ionic liquid with same amount of 2-(2-ethoxyethoxy)ethanol.

In some embodiments, the total amount of tofacitinib reaching at least the epidermis layer provided by the pharmaceutical composition is at least 2 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 3 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 4 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 5 times the total amount of tofacitinib reaching at least the epidermis layer provided by a control composition comprising a PEG-based ointment and 2% of oleyl alcohol.

In some embodiments, the pharmaceutical composition is administered one or more times a day. In some embodiments, the pharmaceutical composition provides reduced systemic exposure to tofacitinib as compared to therapeutically effective doses of oral tofacitinib.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is selected from the group consisting oleic acid, geranic acid, hexanoic acid, and malonic acid. In some embodiments, the fatty acid is geranic acid.

In some embodiments, the ionic liquid is a deep eutectic solvent (DES).

In some embodiments, the ionic liquid comprises the choline cation and fatty acid anion in a molar ratio in a range of 1:1 to 1:4 of choline cation to fatty acid anion. In some embodiments, the ionic liquid comprises the choline cation and fatty acid anion in a molar ratio of 1:2 of choline cation to fatty acid anion.

In some embodiments, the pharmaceutical composition consists essentially of tofacitinib and the ionic liquid. In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an ointment base.

In some embodiments, the ionic liquid is present in the pharmaceutical composition at a concentration of from about 1% to about 99%. In some embodiments, the ionic liquid is present in the pharmaceutical composition at a concentration of from about 5% to about 90%. In some embodiments, the ionic liquid is present in the pharmaceutical composition at a concentration of from about 10% to about 80%. In some embodiments, the ionic liquid is present in the pharmaceutical composition at a concentration of from about 15% to about 70%. In some embodiments, the ionic liquid is present in the pharmaceutical composition at a concentration of from about 20% to about 60%. In some embodiments, the ionic liquid is present in the pharmaceutical composition at a concentration of from about 30% to about 50%. In some embodiments, the ionic liquid is present in the pharmaceutical composition at a concentration of about 35% to about 45%. In some embodiments, the ionic liquid is present in the pharmaceutical composition at a concentration of about 40%.

In some embodiments, the pharmaceutical composition further comprises a penetration enhancer. In some embodiments, the penetration enhancer is 2-(2-ethoxyethoxy)ethanol or oleyl alcohol. In some embodiments, the penetration enhancer is 2-(2-ethoxyethoxy)ethanol. In some embodiments, the pharmaceutical composition comprises from about 1% to about 20% of 2-(2-ethoxyethoxy)ethanol. In some embodiments, the pharmaceutical composition comprises from about 5% to about 15% 2-(2-ethoxyethoxy)ethanol. In some embodiments, the pharmaceutical composition comprises from about 10% 2-(2-ethoxyethoxy)ethanol.

In some embodiments, the pharmaceutical composition is essentially free of 2-(2-ethoxyethoxy)ethanol. In some embodiments, the pharmaceutical composition is essentially free of oleyl alcohol.

### JAK Inhibition

Disclosed herein, in certain embodiments, is a method of locally inhibiting activity of at least one of JAK-1, JAK-2, TYK-2, and JAK-3 within an epidermis layer, a dermis layer, a subcutaneous tissue layer, or a muscle tissue, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

In some embodiments, the pharmaceutical composition comprises about 0.01% to about 10% of tofacitinib or a pharmaceutically acceptable salt thereof. In some embodiments, the pharmaceutical composition comprises about 0.1% to about 8% of tofacitinib or a pharmaceutically acceptable salt thereof. In some embodiments, the topical ointment comprises about 0.1% to about 5% of tofacitinib or a pharmaceutically acceptable salt thereof.

In some embodiments, the target depth is from about 0.05 mm to about 20 mm. In some embodiments, the target depth is from about 0.1 mm to about 15 mm. In some embodiments, the target depth is from about 1 mm to about 10 mm.

In some embodiments, the pharmaceutical composition allows therapeutically effective amount of tofacitinib to reach at least the epidermis layer. In some embodiments, the amount of tofacitinib at the epidermis layer is sufficient to inhibit activity of at least one of JAK-1, JAK-2, TYK-2, and JAK-3 within the epidermis layer.

In some embodiments, the pharmaceutical composition allows therapeutically effective amount of tofacitinib to reach at least the dermis layer. In some embodiments, the amount of tofacitinib at the dermis layer is sufficient to inhibit activity of at least one of JAK-1, JAK-2, TYK-2, and JAK-3 within the dermis layer.

In some embodiments, the pharmaceutical composition allows therapeutically effective amount of tofacitinib to reach at least the the subcutaneous tissue layer. In some embodiments, the amount of tofacitinib at the subcutaneous tissue layer is sufficient to inhibit activity of at least one of JAK-1, JAK-2, TYK-2, and JAK-3 within the subcutaneous tissue layer.

In some embodiments, the pharmaceutical composition allows therapeutically effective amount of tofacitinib to reach at least the muscle tissue. In some embodiments, the amount of tofacitinib at the muscle tissue is sufficient to inhibit activity of at least one of JAK-1, JAK-2, TYK-2, and JAK-3 within the muscle tissue.

In some embodiments, the total amount of tofacitinib reaching at least the epidermis layer provided by the pharmaceutical composition is at least 1.5 times the total amount of tofacitinib reaching at least the epidermis layer provided by a control composition replacing the ionic liquid with same amount of 2-(2-ethoxyethoxy)ethanol. In some embodiments, the total amount of tofacitinib reaching at least the epidermis layer provided by the pharmaceutical composition is at least 2 times the total amount of tofacitinib reaching at least the epidermis layer provided by a control composition replacing the ionic liquid with same amount of 2-(2-ethoxyethoxy)ethanol. In some embodiments, the total amount of tofacitinib reaching at least the epidermis layer provided by the pharmaceutical composition is at least 2.5 times the total amount of tofacitinib reaching at least the epidermis layer provided by a control composition replacing the ionic liquid with same amount of 2-(2-ethoxyethoxy)ethanol. In some embodiments, the total amount of tofacitinib reaching at least the epidermis layer provided by the pharmaceutical composition is at least 3 times the total amount of tofacitinib reaching at least the epidermis layer provided by a control composition replacing the ionic liquid with same amount of 2-(2-ethoxyethoxy)ethanol.

In some embodiments, the total amount of tofacitinib reaching at least the epidermis layer provided by the pharmaceutical composition is at least 2 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 3 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 4 times the total amount of tofacitinib reaching at least the epidermis layer provided by a control composition comprising a PEG-based ointment and 2% of oleyl alcohol. In some embodiments, the total amount of tofacitinib reaching at least the epidermis layer provided by the pharmaceutical composition is at least 5 times the total amount of tofacitinib reaching at least the epidermis layer provided by a control composition comprising a PEG-based ointment and 2% of oleyl alcohol.

In some embodiments, the pharmaceutical composition is administered one or more times a day. In some embodiments, the pharmaceutical composition provides reduced systemic exposure to tofacitinib as compared to therapeutically effective doses of oral tofacitinib.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is selected from the group consisting oleic acid, geranic acid, hexanoic acid, and malonic acid. In some embodiments, the fatty acid is geranic acid.

In some embodiments, the ionic liquid is a deep eutectic solvent (DES). In some embodiments, the ionic liquid comprises the choline cation and fatty acid anion in a molar ratio in a range of 1:1 to 1:4 of choline cation to fatty acid anion. In some embodiments, the ionic liquid comprises the choline cation and fatty acid anion in a molar ratio of 1:2 of choline cation to fatty acid anion.

In some embodiments, the pharmaceutical composition consists essentially of tofacitinib and the ionic liquid. In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an ointment base.

In some embodiments, the ionic liquid is present in the pharmaceutical composition at a concentration of from about 1% to about 99%. In some embodiments, the ionic liquid is present in the pharmaceutical composition at a concentration of from about 5% to about 90%. In some embodiments, the ionic liquid is present in the pharmaceutical composition at a concentration of from about 10% to about 80%. In some embodiments, the ionic liquid is present in the pharmaceutical composition at a concentration of from about 15% to about 70%. In some embodiments, the ionic liquid is present in the pharmaceutical composition at a concentration of from about 20% to about 60%. In some embodiments, the ionic liquid is present in the pharmaceutical composition at a concentration of from about 30% to about 50%. In some embodiments, the ionic liquid is present in the pharmaceutical composition at a concentration of about 35% to about 45. In some embodiments, the ionic liquid is present in the pharmaceutical composition at a concentration of about 40%.

In some embodiments, the pharmaceutical composition further comprises a penetration enhancer. In some embodiments, the penetration enhancer is 2-(2-ethoxyethoxy)ethanol or oleyl alcohol. In some embodiments, the penetration enhancer is 2-(2-ethoxyethoxy)ethanol. In some embodiments, the pharmaceutical composition comprises from about 1% to about 20% of 2-(2-ethoxyethoxy)ethanol. In some embodiments, the pharmaceutical composition comprises from about 5% to about 15% 2-(2-ethoxyethoxy)ethanol. In some embodiments, the pharmaceutical composition comprises from about 10% 2-(2-ethoxyethoxy)ethanol.

In some embodiments, the pharmaceutical composition is essentially free of 2-(2-ethoxyethoxy)ethanol. In some embodiments, the pharmaceutical composition is essentially free of oleyl alcohol.

In some embodiments, the amount of the composition administered to the individual and the length of treatment depends on the attributes of the individual including, but not limited to, state of health, weight, severity of the condition, previous therapy, and judgement of the treating physician. In some embodiments, the amount of the composition administered to the individual is determined by routine experimentation (e.g., a dose escalation clinical trial).

In some embodiments, topical compositions as described herein inhibits activity of at least one of JAK-1, JAK-2, TYK-2, and JAK-3 within the epidermis layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of at least one of JAK-1, JAK-2, TYK-2, and JAK-3 within the dermis layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of at least one of JAK-1, JAK-2, TYK-2, and JAK-3 within the subcutaneous tissue layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of at least one of JAK-1, JAK-2, TYK-2, and JAK-3 within the muscle by 10%, 15%, 20%, 25%, 30%, 35%, 40%, 45%, 50%, 55%, 60%, 65%, 70%, 75%, 80%, 85%, 90%, 95%, or more than 95%.

In some embodiments, the composition is applied to the skin of the individual once a day. In some embodiments, the composition is applied to the skin of the individual 1, 2, 3, 4, or 5 times a day. In some embodiments, the composition is applied to the skin of the individual 2 times a day. In some embodiments, the composition is applied to the skin of the individual 2 times a day, e.g., morning and evening. In some embodiments, the composition is applied to the skin of the individual every day, every other day, every three days, twice a week, once a week, or once a month. In some embodiments, the composition is applied to the skin of the individual once. In some embodiments, the composition is applied to the skin of the individual for a period of time of 1 week, 2 weeks, 3 weeks, 1 month, 2 months, or 3 months or more. In some embodiments, the composition is applied to the skin until the symptoms of the disease or condition associated with activity of JAK(s), such as JAK-1, JAK-2, TYK-2, and/or JAK-3, are eliminated. In some embodiments, the composition is applied to the skin until the symptoms of the disease or condition associated with activity of JAK(s), such as JAK-1, JAK-2, TYK-2, and/or JAK-3, are reduced.

In some embodiments, the topical tofacitinib composition described herein provides improved stability or less degradation of the tofacitinib therein. In some embodiments, the [0088] In some embodiments, the topical tofacitinib composition described herein provides improved stability or less degradation of the tofacitinib therein. In some embodiments, the topical tofacitinib composition described herein are stable with respect to compound degradation (e.g. less than 30% degradation, less than 25% degradation, less than 20% degradation, less than 15% degradation, less than 10% degradation, less than 8% degradation, less than 5% degradation, less than 3% degradation, less than 2% degradation, or less than 1% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months under storage conditions (e.g. room temperature). In other embodiments, the formulations described herein are stable with respect to tofacitinib degradation over a period of at least about 1 week. Also described herein are formulations that are stable with respect to tofacitinib degradation over a period of at least about 1 month.

### Certain Non-Limiting Embodiments

Embodiment 1. A method of delivering tofacitinib to or through a skin, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

Embodiment 2. The method of Embodiment 1, wherein the pharmaceutical composition comprises about 0.01% to about 10% of tofacitinib or a pharmaceutically acceptable salt thereof.

Embodiment 3. The method of Embodiment 1, wherein the pharmaceutical composition comprises about 0.1% to about 8% of tofacitinib or a pharmaceutically acceptable salt thereof.

Embodiment 4. The method of Embodiment 1, wherein the pharmaceutical composition comprises about 0.1% to about 5% of tofacitinib or a pharmaceutically acceptable salt thereof.

Embodiment 5. The method of any one of Embodiments 1-4, wherein the target depth is from about 0.05 mm to about 20 mm.

Embodiment 6. The method of any one of Embodiments 1-4, wherein the target depth is from about 0.1 mm to about 15 mm.

Embodiment 7. The method of any one of Embodiments 1-4, wherein the target depth is from about 1 mm to about 10 mm.

Embodiment 8. The method of any one of Embodiments 1-7, wherein the pharmaceutical composition allows therapeutically effective amount of tofacitinib to reach at least an epidermis layer.

Embodiment 9. The method of Embodiment 8, wherein the amount of tofacitinib at the epidermis layer is sufficient to inhibit activity of at least one of JAK-1, JAK-2, TYK-2, and JAK-3 within the epidermis layer.

Embodiment 10. The method of any one of Embodiments 1-9, wherein the pharmaceutical composition allows therapeutically effective amount of tofacitinib to reach at least a dermis layer.

Embodiment 11. The method of Embodiment 10, wherein the amount of tofacitinib at the dermis layer is sufficient to inhibit activity of at least one of JAK-1, JAK-2, TYK-2, and JAK-3 within the dermis layer.

Embodiment 12. The method of any one of Embodiments 1-11, wherein the pharmaceutical composition allows therapeutically effective amount of tofacitinib to reach at least the a subcutaneous tissue layer.

Embodiment 13. The method of Embodiment 12, wherein the amount of tofacitinib at the subcutaneous tissue layer is sufficient to inhibit activity of at least one of JAK-1, JAK-2, TYK-2, and JAK-3 within the subcutaneous tissue layer.

Embodiment 14. The method of any one of Embodiments 1-13, wherein the pharmaceutical composition allows therapeutically effective amount of tofacitinib to reach at least a muscle tissue.

Embodiment 15. The method of Embodiment 14, wherein the amount of tofacitinib at the muscle tissue is sufficient to inhibit activity of at least one of JAK-1, JAK-2, TYK-2, and JAK-3 within the muscle tissue.

Embodiment 16. The method of any one of Embodiments 1-15, wherein the total amount of tofacitinib reaching at least the epidermis layer provided by the pharmaceutical composition is at least 1.5 times the total amount of tofacitinib reaching at least the epidermis layer provided by a control composition replacing the ionic liquid with same amount of 2-(2-ethoxyethoxy)ethanol.

Embodiment 17. The method of any one of Embodiments 1-15, wherein the total amount of tofacitinib reaching at least the epidermis layer provided by the pharmaceutical composition is at least 2 times the total amount of tofacitinib reaching at least the epidermis layer provided by a control composition replacing the ionic liquid with same amount of 2-(2-ethoxyethoxy)ethanol.

Embodiment 18. The method of any one of Embodiments 1-15, wherein the total amount of tofacitinib reaching at least the epidermis layer provided by the pharmaceutical composition is at least 2.5 times the total amount of tofacitinib reaching at least the epidermis layer provided by a control composition replacing the ionic liquid with same amount of 2-(2-ethoxyethoxy)ethanol.

Embodiment 19. The method of any one of Embodiments 1-15, wherein the total amount of tofacitinib reaching at least the epidermis layer provided by the pharmaceutical composition is at least 3 times the total amount of tofacitinib reaching at least the epidermis layer provided by a control composition replacing the ionic liquid with same amount of 2-(2-ethoxyethoxy)ethanol.

Embodiment 20. The method of any one of Embodiments 1-19, wherein the total amount of tofacitinib reaching at least the epidermis layer provided by the pharmaceutical composition is at least 2 times the total amount of tofacitinib reaching at least the epidermis layer provided by a control composition comprising a PEG-based ointment and 2% of oleyl alcohol.

Embodiment 21. The method of any one of Embodiments 1-19, wherein the total amount of tofacitinib reaching at least the epidermis layer provided by the pharmaceutical composition is at least 3 times the total amount of tofacitinib reaching at least the epidermis layer provided by a control composition comprising a PEG-based ointment and 2% of oleyl alcohol.

Embodiment 22. The method of any one of Embodiments 1-19, wherein the total amount of tofacitinib reaching at least the epidermis layer provided by the pharmaceutical composition is at least 4 times the total amount of tofacitinib reaching at least the epidermis layer provided by a control composition comprising a PEG-based ointment and 2% of oleyl alcohol.

Embodiment 23. The method of any one of Embodiments 1-19, wherein the total amount of tofacitinib reaching at least the epidermis layer provided by the pharmaceutical composition is at least 5 times the total amount of tofacitinib reaching at least the epidermis layer provided by a control composition comprising a PEG-based ointment and 2% of oleyl alcohol.

Embodiment 24. The method of any one of Embodiments 1-23, wherein the pharmaceutical composition is administered one or more times a day.

Embodiment 25. The method of any one of Embodiments 1-24, wherein the pharmaceutical composition provides reduced systemic exposure to tofacitinib as compared to therapeutically effective doses of oral tofacitinib.

Embodiment 26. The method of any one of Embodiments 1-25, wherein the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.

Embodiment 27. The method of any one of Embodiments 1-25, wherein the fatty acid is selected from the group consisting oleic acid, geranic acid, hexanoic acid, and malonic acid.

Embodiment 28. The method of any one of Embodiments 1-25, wherein the fatty acid is geranic acid.

Embodiment 29. The method of any one of Embodiments 1-28, wherein the ionic liquid is a deep eutectic solvent (DES).

Embodiment 30. The method of any one of Embodiments 1-29, wherein the ionic liquid comprises the choline cation and fatty acid anion in a molar ratio in a range of 1:1 to 1:4 of choline cation to fatty acid anion.

Embodiment 31. The method of any one of Embodiments 1-29, wherein the ionic liquid comprises the choline cation and fatty acid anion in a molar ratio of 1:2 of choline cation to fatty acid anion.

Embodiment 32. The method of any one of Embodiments 1-30, wherein the pharmaceutical composition consists essentially of tofacitinib and the ionic liquid.

Embodiment 33. The method of any one of Embodiments 1-30, wherein the pharmaceutical composition further comprises a pharmaceutically acceptable carrier.

Embodiment 34. The method of Embodiment 33, wherein the pharmaceutically acceptable carrier is an aqueous carrier.

Embodiment 35. The method of Embodiment 33, wherein the pharmaceutically acceptable carrier comprises an ointment base.

Embodiment 36. The method of any one of Embodiments 1-35, wherein the ionic liquid is present in the pharmaceutical composition at a concentration of from about 1% to about 99%.

Embodiment 37. The method of any one of Embodiments 1-35, wherein the ionic liquid is present in the pharmaceutical composition at a concentration of from about 5% to about 90%.

Embodiment 38. The method of any one of Embodiments 1-35, wherein the ionic liquid is present in the pharmaceutical composition at a concentration of from about 10% to about 80%.

Embodiment 39. The method of any one of Embodiments 1-35, wherein the ionic liquid is present in the pharmaceutical composition at a concentration of from about 15% to about 70%.

Embodiment 40. The method of any one of Embodiments 1-35, wherein the ionic liquid is present in the pharmaceutical composition at a concentration of from about 20% to about 60%.

Embodiment 41. The method of any one of Embodiments 1-35, wherein the ionic liquid is present in the pharmaceutical composition at a concentration of from about 30% to about 50%.

Embodiment 42. The method of any one of Embodiments 1-35, wherein the ionic liquid is present in the pharmaceutical composition at a concentration of about 35% to about 45%.

Embodiment 43. The method of any one of Embodiments 1-35, wherein the ionic liquid is present in the pharmaceutical composition at a concentration of about 40%.

Embodiment 44. The method of any one of Embodiments 1-43, wherein the pharmaceutical composition further comprises a penetration enhancer.

Embodiment 45. The method of Embodiment 44, wherein the penetration enhancer is 2-(2-ethoxyethoxy)ethanol or oleyl alcohol.

Embodiment 46. The method of Embodiment 44, wherein the penetration enhancer is 2-(2-ethoxyethoxy)ethanol.

Embodiment 47. The method of Embodiment 46, wherein the pharmaceutical composition comprises from about 1% to about 20% of 2-(2-ethoxyethoxy)ethanol.

Embodiment 48. The method of Embodiment 46, wherein the pharmaceutical composition comprises from about 5% to about 15% 2-(2-ethoxyethoxy)ethanol.

Embodiment 49. The method of any one of Embodiments 46, wherein the pharmaceutical composition comprises from about 10% 2-(2-ethoxyethoxy)ethanol.

Embodiment 50. The method of any one of Embodiments 1-43, wherein the pharmaceutical composition is essentially free of 2-(2-ethoxyethoxy)ethanol or oleyl alcohol.

Embodiment 51. A method of locally inhibiting activity of at least one of JAK-1, JAK-2, TYK-2, and JAK-3 within an epidermis layer, a dermis layer, a subcutaneous tissue layer, or a muscle tissue, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

Embodiment 52. The method of Embodiment 51, wherein the pharmaceutical composition comprises about 0.01% to about 10% of tofacitinib or a pharmaceutically acceptable salt thereof.

Embodiment 53. The method of Embodiment 51, wherein the pharmaceutical composition comprises about 0.1% to about 8% of tofacitinib or a pharmaceutically acceptable salt thereof.

Embodiment 54. The method of Embodiment 51, wherein the pharmaceutical composition comprises about 0.1% to about 5% of tofacitinib or a pharmaceutically acceptable salt thereof.

Embodiment 55. The method of any one of Embodiments 51-54, wherein the target depth is from about 0.05 mm to about 20 mm.

Embodiment 56. The method of any one of Embodiments 51-54, wherein the target depth is from about 0.1 mm to about 15 mm.

Embodiment 57. The method of any one of Embodiments 51-54, wherein the target depth is from about 1 mm to about 10 mm.

Embodiment 58. The method of any one of Embodiments 51-57, wherein the pharmaceutical composition allows therapeutically effective amount of tofacitinib to reach at least the epidermis layer.

Embodiment 59. The method of Embodiment 58, wherein the amount of tofacitinib at the epidermis layer is sufficient to inhibit activity of at least one of JAK-1, JAK-2, TYK-2, and JAK-3 within the epidermis layer.

Embodiment 60. The method of any one of Embodiments 51-59, wherein the pharmaceutical composition allows therapeutically effective amount of tofacitinib to reach at least the dermis layer.

Embodiment 61. The method of Embodiment 60, wherein the amount of tofacitinib at the dermis layer is sufficient to inhibit activity of at least one of JAK-1, JAK-2, TYK-2, and JAK-3 within the dermis layer.

Embodiment 62. The method of any one of Embodiments 51-61, wherein the pharmaceutical composition allows therapeutically effective amount of tofacitinib to reach at least the the subcutaneous tissue layer.

Embodiment 63. The method of Embodiment 62, wherein the amount of tofacitinib at the subcutaneous tissue layer is sufficient to inhibit activity of at least one of JAK-1, JAK-2, TYK-2, and JAK-3 within the subcutaneous tissue layer.

Embodiment 64. The method of any one of Embodiments 51-63, wherein the pharmaceutical composition allows therapeutically effective amount of tofacitinib to reach at least the muscle tissue.

Embodiment 65. The method of Embodiment 64, wherein the amount of tofacitinib at the muscle tissue is sufficient to inhibit activity of at least one of JAK-1, JAK-2, TYK-2, and JAK-3 within the muscle tissue.

Embodiment 66. The method of any one of Embodiments 51-65, wherein the total amount of tofacitinib reaching at least the epidermis layer provided by the pharmaceutical composition is at least 1.5 times the total amount of tofacitinib reaching at least the epidermis layer provided by a control composition replacing the ionic liquid with same amount of 2-(2-ethoxyethoxy)ethanol.

Embodiment 67. The method of any one of Embodiments 51-65, wherein the total amount of tofacitinib reaching at least the epidermis layer provided by the pharmaceutical composition is at least 2 times the total amount of tofacitinib reaching at least the epidermis layer provided by a control composition replacing the ionic liquid with same amount of 2-(2-ethoxyethoxy)ethanol.

Embodiment 68. The method of any one of Embodiments 51-65, wherein the total amount of tofacitinib reaching at least the epidermis layer provided by the pharmaceutical composition is at least 2.5 times the total amount of tofacitinib reaching at least the epidermis layer provided by a control composition replacing the ionic liquid with same amount of 2-(2-ethoxyethoxy)ethanol.

Embodiment 69. The method of any one of Embodiments 51-65, wherein the total amount of tofacitinib reaching at least the epidermis layer provided by the pharmaceutical composition is at least 3 times the total amount of tofacitinib reaching at least the epidermis layer provided by a control composition replacing the ionic liquid with same amount of 2-(2-ethoxyethoxy)ethanol.

Embodiment 70. The method of any one of Embodiments 51-69, wherein the total amount of tofacitinib reaching at least the epidermis layer provided by the pharmaceutical composition is at least 2 times the total amount of tofacitinib reaching at least the epidermis layer provided by a control composition comprising a PEG-based ointment and 2% of oleyl alcohol.

Embodiment 71. The method of any one of Embodiments 51-69, wherein the total amount of tofacitinib reaching at least the epidermis layer provided by the pharmaceutical composition is at least 3 times the total amount of tofacitinib reaching at least the epidermis layer provided by a control composition comprising a PEG-based ointment and 2% of oleyl alcohol.

Embodiment 72. The method of any one of Embodiments 51-69, wherein the total amount of tofacitinib reaching at least the epidermis layer provided by the pharmaceutical composition is at least 4 times the total amount of tofacitinib reaching at least the epidermis layer provided by a control composition comprising a PEG-based ointment and 2% of oleyl alcohol.

Embodiment 73. The method of any one of Embodiments 51-69, wherein the total amount of tofacitinib reaching at least the epidermis layer provided by the pharmaceutical composition is at least 5 times the total amount of tofacitinib reaching at least the epidermis layer provided by a control composition comprising a PEG-based ointment and 2% of oleyl alcohol.

Embodiment 74. The method of any one of Embodiments 51-73, wherein the pharmaceutical composition is administered one or more times a day.

Embodiment 75. The method of any one of Embodiments 51-74, wherein the pharmaceutical composition provides reduced systemic exposure to tofacitinib as compared to therapeutically effective doses of oral tofacitinib.

Embodiment 76. The method of any one of Embodiments 51-75, wherein the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.

Embodiment 77. The method of any one of Embodiments 51-75, wherein the fatty acid is selected from the group consisting oleic acid, geranic acid, hexanoic acid, and malonic acid.

Embodiment 78. The method of any one of Embodiments 51-75, wherein the fatty acid is geranic acid.

Embodiment 79. The method of any one of Embodiments 51-78, wherein the ionic liquid is a deep eutectic solvent (DES).

Embodiment 80. The method of any one of Embodiments 51-79, wherein the ionic liquid comprises the choline cation and fatty acid anion in a molar ratio in a range of 1:1 to 1:4 of choline cation to fatty acid anion.

Embodiment 81. The method of any one of Embodiments 51-79, wherein the ionic liquid comprises the choline cation and fatty acid anion in a molar ratio of 1:2 of choline cation to fatty acid anion.

Embodiment 82. The method of any one of Embodiments 51-80, wherein the pharmaceutical composition consists essentially of tofacitinib and the ionic liquid.

Embodiment 83. The method of any one of Embodiments 51-80, wherein the pharmaceutical composition further comprises a pharmaceutically acceptable carrier.

Embodiment 84. The method of Embodiment 83, wherein the pharmaceutically acceptable carrier is an aqueous carrier.

Embodiment 85. The method of Embodiment 83, wherein the pharmaceutically acceptable carrier comprises an ointment base.

Embodiment 86. The method of any one of Embodiments 51-85, wherein the ionic liquid is present in the pharmaceutical composition at a concentration of from about 1% to about 99%.

Embodiment 87. The method of any one of Embodiments 51-85, wherein the ionic liquid is present in the pharmaceutical composition at a concentration of from about 5% to about 90%.

Embodiment 88. The method of any one of Embodiments 51-85, wherein the ionic liquid is present in the pharmaceutical composition at a concentration of from about 10% to about 80%.

Embodiment 89. The method of any one of Embodiments 51-85, wherein the ionic liquid is present in the pharmaceutical composition at a concentration of from about 15% to about 70%.

Embodiment 90. The method of any one of Embodiments 51-85, wherein the ionic liquid is present in the pharmaceutical composition at a concentration of from about 20% to about 60%.

Embodiment 91. The method of any one of Embodiments 51-85, wherein the ionic liquid is present in the pharmaceutical composition at a concentration of from about 30% to about 50%.

Embodiment 92. The method of any one of Embodiments 51-85, wherein the ionic liquid is present in the pharmaceutical composition at a concentration of about 35% to about 40%.

Embodiment 93. The method of any one of Embodiments 51-85, wherein the ionic liquid is present in the pharmaceutical composition at a concentration of about 40%.

Embodiment 94. The method of any one of Embodiments 51-93, wherein the pharmaceutical composition further comprises a penetration enhancer.

Embodiment 95. The method of Embodiment 94, wherein the penetration enhancer is 2-(2-ethoxyethoxy)ethanol or oleyl alcohol.

Embodiment 96. The method of Embodiment 94, wherein the penetration enhancer is 2-(2-ethoxyethoxy)ethanol.

Embodiment 97. The method of Embodiment 96, wherein the pharmaceutical composition comprises from about 1% to about 20% of 2-(2-ethoxyethoxy)ethanol.

Embodiment 98. The method of Embodiment 96, wherein the pharmaceutical composition comprises from about 5% to about 15% 2-(2-ethoxyethoxy)ethanol.

Embodiment 99. The method of Embodiment 96, wherein the pharmaceutical composition comprises from about 10% 2-(2-ethoxyethoxy)ethanol.

Embodiment 100. The method of any one of Embodiments 51-93, wherein the pharmaceutical composition is essentially free of 2-(2-ethoxyethoxy)ethanol or oleyl alcohol.

Embodiment 101. A topical composition, comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

102. The composition of Embodiment 101, wherein the pharmaceutical composition comprises about 0.01% to about 10% of tofacitinib or a pharmaceutically acceptable salt thereof.

Embodiment 103. The composition of Embodiment 101, wherein the pharmaceutical composition comprises about 0.1% to about 8% of tofacitinib or a pharmaceutically acceptable salt thereof.

Embodiment 104. The composition of Embodiment 101, wherein the pharmaceutical composition comprises about 0.1% to about 5% of tofacitinib or a pharmaceutically acceptable salt thereof.

Embodiment 105. The composition of any one of Embodiments 101-104, wherein the target depth is from about 0.05 mm to about 20 mm.

Embodiment 106. The composition of any one of Embodiments 101-104, wherein the target depth is from about 0.1 mm to about 15 mm.

Embodiment 107. The composition of any one of Embodiments 101-104, wherein the target depth is from about 1 mm to about 10 mm.

Embodiment 108. The composition of any one of Embodiments 101-107, wherein the pharmaceutical composition allows therapeutically effective amount of tofacitinib to reach at least an epidermis layer.

Embodiment 109. The composition of any one of Embodiments 101-108, wherein the pharmaceutical composition allows therapeutically effective amount of tofacitinib to reach at least a dermis layer.

Embodiment 110. The composition of any one of Embodiments 101-109, wherein the pharmaceutical composition allows therapeutically effective amount of tofacitinib to reach at least a subcutaneous tissue layer.

Embodiment 111. The composition of any one of Embodiments 101-110, wherein the pharmaceutical composition allows therapeutically effective amount of tofacitinib to reach at least a muscle tissue.

Embodiment 112. The composition of any one of Embodiments 101-111, wherein the total amount of tofacitinib reaching at least the epidermis layer provided by the pharmaceutical composition is at least 1.5 times the total amount of tofacitinib reaching at least the epidermis layer provided by a control composition replacing the ionic liquid with same amount of 2-(2-ethoxyethoxy)ethanol.

Embodiment 113. The composition of any one of Embodiments 101-111, wherein the total amount of tofacitinib reaching at least the epidermis layer provided by the pharmaceutical composition is at least 2 times the total amount of tofacitinib reaching at least the epidermis layer provided by a control composition replacing the ionic liquid with same amount of 2-(2-ethoxyethoxy)ethanol.

Embodiment 114. The composition of any one of Embodiments 101-111, wherein the total amount of tofacitinib reaching at least the epidermis layer provided by the pharmaceutical composition is at least 2.5 times the total amount of tofacitinib reaching at least the epidermis layer provided by a control composition replacing the ionic liquid with same amount of 2-(2-ethoxyethoxy)ethanol.

Embodiment 115. The composition of any one of Embodiments 101-111, wherein the total amount of tofacitinib reaching at least the epidermis layer provided by the pharmaceutical composition is at least 3 times the total amount of tofacitinib reaching at least the epidermis layer provided by a control composition replacing the ionic liquid with same amount of 2-(2-ethoxyethoxy)ethanol.

Embodiment 116. The composition of any one of Embodiments 101-115, wherein the total amount of tofacitinib reaching at least the epidermis layer provided by the pharmaceutical composition is at least 2 times the total amount of tofacitinib reaching at least the epidermis layer provided by a control composition comprising a PEG-based ointment and 2% of oleyl alcohol.

Embodiment 117. The composition of any one of Embodiments 101-115, wherein the total amount of tofacitinib reaching at least the epidermis layer provided by the pharmaceutical composition is at least 3 times the total amount of tofacitinib reaching at least the epidermis layer provided by a control composition comprising a PEG-based ointment and 2% of oleyl alcohol.

Embodiment 118. The composition of any one of Embodiments 101-115, wherein the total amount of tofacitinib reaching at least the epidermis layer provided by the pharmaceutical composition is at least 4 times the total amount of tofacitinib reaching at least the epidermis layer provided by a control composition comprising a PEG-based ointment and 2% of oleyl alcohol.

Embodiment 119. The composition of any one of Embodiments 101-115, wherein the total amount of tofacitinib reaching at least the epidermis layer provided by the pharmaceutical composition is at least 5 times the total amount of tofacitinib reaching at least the epidermis layer provided by a control composition comprising a PEG-based ointment and 2% of oleyl alcohol.

Embodiment 120. The composition of any one of Embodiments 101-119, wherein the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.

Embodiment 121. The composition of any one of Embodiments 101-119, wherein the fatty acid is selected from the group consisting oleic acid, geranic acid, hexanoic acid, and malonic acid.

Embodiment 122. The composition of any one of Embodiments 101-119, wherein the fatty acid is geranic acid.

Embodiment 123. The composition of any one of Embodiments 101-122, wherein the ionic liquid is a deep eutectic solvent (DES).

Embodiment 124. The composition of any one of Embodiments 101-123, wherein the ionic liquid comprises the choline cation and fatty acid anion in a molar ratio in a range of 1:1 to 1:4 of choline cation to fatty acid anion.

Embodiment 125. The composition of any one of Embodiments 101-123, wherein the ionic liquid comprises the choline cation and fatty acid anion in a molar ratio of 1:2 of choline cation to fatty acid anion.

Embodiment 126. The composition of any one of Embodiments 101-125, wherein the pharmaceutical composition consists essentially of tofacitinib and the ionic liquid.

Embodiment 127. The composition of any one of Embodiments 101-125, wherein the pharmaceutical composition further comprises a pharmaceutically acceptable carrier.

Embodiment 128. The composition of Embodiment 127, wherein the pharmaceutically acceptable carrier is an aqueous carrier.

Embodiment 129. The composition of Embodiment 127, wherein the pharmaceutically acceptable carrier comprises an ointment base.

Embodiment 130. The composition of any one of Embodiments 101-129, wherein the ionic liquid is present in the pharmaceutical composition at a concentration of from about 1% to about 99%.

Embodiment 131. The composition of any one of Embodiments 101-129, wherein the ionic liquid is present in the pharmaceutical composition at a concentration of from about 5% to about 90%.

Embodiment 132. The composition of any one of Embodiments 101-129, wherein the ionic liquid is present in the pharmaceutical composition at a concentration of from about 10% to about 80%.

Embodiment 133. The composition of any one of Embodiments 101-129, wherein the ionic liquid is present in the pharmaceutical composition at a concentration of from about 15% to about 70%.

Embodiment 134. The composition of any one of Embodiments 101-129, wherein the ionic liquid is present in the pharmaceutical composition at a concentration of from about 20% to about 60%.

Embodiment 135. The composition of any one of Embodiments 101-129, wherein the ionic liquid is present in the pharmaceutical composition at a concentration of from about 30% to about 50%.

Embodiment 136. The composition of any one of Embodiments 101-129, wherein the ionic liquid is present in the pharmaceutical composition at a concentration of about 35% to about 45%.

Embodiment 137. The composition of any one of Embodiments 101-129, wherein the ionic liquid is present in the pharmaceutical composition at a concentration of about 40%.

Embodiment 138. The composition of any one of Embodiments 101-137, wherein the pharmaceutical composition further comprises a penetration enhancer.

Embodiment 139. The composition of Embodiment 138, wherein the penetration enhancer is 2-(2-ethoxyethoxy)ethanol or oleyl alcohol.

Embodiment 140. The composition of Embodiment 138, wherein the penetration enhancer is 2-(2-ethoxyethoxy)ethanol.

Embodiment 141. The composition of Embodiment 140, wherein the pharmaceutical composition comprises from about 1% to about 20% of 2-(2-ethoxyethoxy)ethanol.

Embodiment 142. The composition of Embodiment 140, wherein the pharmaceutical composition comprises from about 5% to about 15% 2-(2-ethoxyethoxy)ethanol.

Embodiment 143. The composition of Embodiment 140, wherein the pharmaceutical composition comprises from about 10% 2-(2-ethoxyethoxy)ethanol.

Embodiment 144. The composition of any one of Embodiments 101-137, wherein the pharmaceutical composition is essentially free of 2-(2-ethoxyethoxy)ethanol or oleyl alcohol.

### Certain Terminology

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for rosacea, if, after receiving a therapeutic amount of a composition according to the methods of the present disclosure, the subject shows observable and/or measurable reduction in or absence of one or more of the following: reduction in the erythema; reduction in the appearance of red veins; papules, and pustules.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### NON-LIMITING EXAMPLES

### Example 1: Preparation of an Ionic Liquid Containing Choline Cation and Geranic Acid Anion in a 1:1 Molar Ratio (Ionic Liquid B)

The purified GMP Penta Geranic acid (311.0 g, 1.848 mol) was placed in a 2 L round bottomed flask. The flask was placed in a water bath at 20° C. and stirred. Then choline bicarbonate (381.7 g, 1.848 mol) 80% solution in water (Sigma, C7519, 209 ml) was added slowly (drop-wise) with an addition funnel, total addition time was 120 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100 kPa for 36 additional hrs to dry the final product. 475 g of product (94.7% yield) was obtained. HPLC analysis shows 97.9% purity.

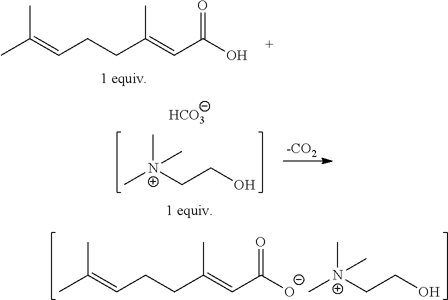

### Example 2: Preparation of an Ionic Liquid Containing Choline Cation and Geranic Acid Anion in a 1:2 Molar Ratio (Ionic Liquid A)

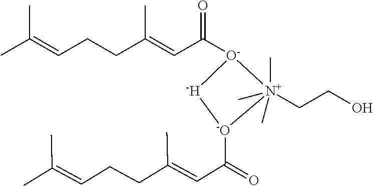

To two equivalents (9.88 g., 0.059 moles) of neat geranic acid, recrystallized 5× at −70° C. from 70% geranic acid/30% acetone, in a 500 mL round bottom flask was added one equivalent of choline bicarbonate (80 wt % solution, 6.06 g, 0.029 mol). The mixture was stirred at room temperature until no more CO₂ evolved. Solvent was removed by rotary evaporation at 60° C. for 20 min, and the product was dried in a vacuum oven for 48 h at 60° C.

Physical characterization at 25° C.: solubility in water=0.5 M; density=0.990 g/mL; conductivity=0.0431 mS/cm; viscosity=1345 cP.

### Example 3: Alternate Preparation of an Ionic Liquid Containing Choline Cation and Geranic Acid Anion in a 1:2 Molar Ratio (Ionic Liquid A)

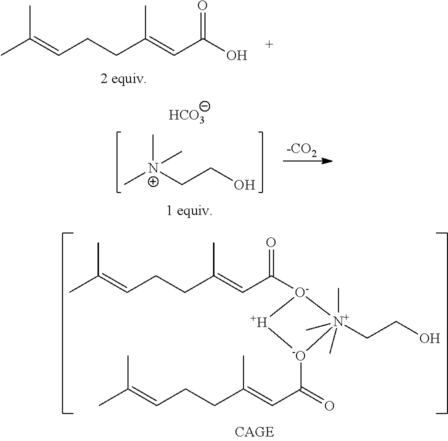

The purified GMP Penta Geranic acid (155 g, 0.921 mol) was placed in a 1 L round bottomed flask. The flask was placed in a water bath at 20° C. and stirred. Then choline bicarbonate (95.1 g, 0.460) 80% solution in water (Sigma, C7519, Lot #: 059K1526V, 209 ml) was added slowly (drop-wise) with an addition funnel, total addition time was 35 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100 kPa for 36 additional hrs to dry the final product. 197 g of Cage (96% yield) was obtained. 1H-NMR spectrum looks similar to the one of CB-0001. HPLC analysis shows 95.1% purity.

### Example 4: Preparation of an Ionic Liquid Containing Choline Cation and Geranic Acid Anion in a 1:3 Molar Ratio

To three equivalents (14.56 g., 0.087 moles) of neat geranic acid, recrystallized 5× at −70° C. from 70% geranic acid/30% acetone, in a 1000 mL round bottom flask is added one equivalent of choline bicarbonate (80 wt % solution, 6.06 g, 0.029 mol). The mixture is stirred at room temperature until no more CO₂ evolved. Solvent is removed by rotary evaporation at 60° C. for 20 min, and the product is dried in a vacuum oven for 48 h at 60° C.

### Example 5: Preparation of an Ionic Liquid Containing Choline and Geranic Acid in a 1:4 Molar Ratio

To four equivalents (19.76 g., 0.118 moles) of neat geranic acid, recrystallized 5× at −70° C. from 70% geranic acid/30% acetone, in a 800 mL round bottom flask is added one equivalent of choline bicarbonate (80 wt % solution, 6.06 g, 0.029 mol). The mixture is stirred at room temperature until no more CO₂ evolved. Solvent is removed by rotary evaporation at 60° C. for 20 min, and the product is dried in a vacuum oven for 48 h at 60° C.

### Example 6: Preparation of a Topical Composition Containing Tofacitinib and Ionic Liquid (Tofacitinib Composition A)

Tofacitinib, Ionic Liquid A (according to Example 2) and water are mixture together until a homogenous composition is achieved.

TABLE 1
Tofacitinib Composition A

| Components | Concentration (w/w) |
|---|---|
| Tofacitinib | 0.5% |
| Ionic Liquid A | 40% |
| Water | 59.5% |

### Example 7: Preparation of a topical composition containing tofacitinib, ionic liquid, and Transcutol® (Tofacitinib Composition B)

Tofacitinib, Ionic Liquid A (according to Example 2), 2-(2-ethoxyethoxy)ethanol (Transcutol®), and water are mixture together until a homogenous composition is achieved.

TABLE 2
Tofacitinib Composition B

| Components | Concentration (w/w) |
|---|---|
| Tofacitinib | 0.5% |
| Ionic Liquid A | 40% |
| Transcutol ® | 10% |
| Water | 49.5% |

### Example 8: Preparation of a Topical Composition Containing Tofacitinib and Transcutol® (Control Composition A)

Tofacitinib, 2-(2-ethoxyethoxy)ethanol (Transcutol®), and water are mixture together until a homogenous composition is achieved.

TABLE 3
Control Composition A

| Components | Concentration (w/w) |
|---|---|
| Tofacitinib | 0.5% |
| Transcutol ® | 40% |
| Water | 59.5% |

### Example 9: Preparation of a Topical Composition Containing Tofacitinib and Oleyl Alcohol in a PEG-Based Ointment (Control Composition B)

A ointment composition containing 0.5% tofacitinib, 2% oleyl alcohol, and an ointment carrier is prepared according to WO2012/137111 (Table 28).

### Example 10: Skin Flux Assay of Tofacitinib Compositions

Tofacitinib delivery by the following four compositions was measured using a static Franz cell setup (N=3 for each composition).

1. Tofacitinib Composition A (N=3)

2. Tofacitinib Composition B (N=3)

3. Control Composition A (N=3)

4. Control Composition B (N=3)

Study was performed in Franz cells with human cadaver skin clamped over receptor chamber containing saline. The skin sample thickness was between 0.24-0.53 mm. The test composition was applied to surface of skin and occluded. After 24 h at 37° C., the surface of the skin sample was washed with saline and tape stripped to remove compositions remaining on the outer surface of the skin sample. The skin sample was then extracted in methanol: saline, 1:1, v/v. Dermis and epidermis layers were extracted together and not separated. Receptor is the saline underneath the skin that was sampled directly. The concentration of tofacitinib in these samples was quantified by HPLC.

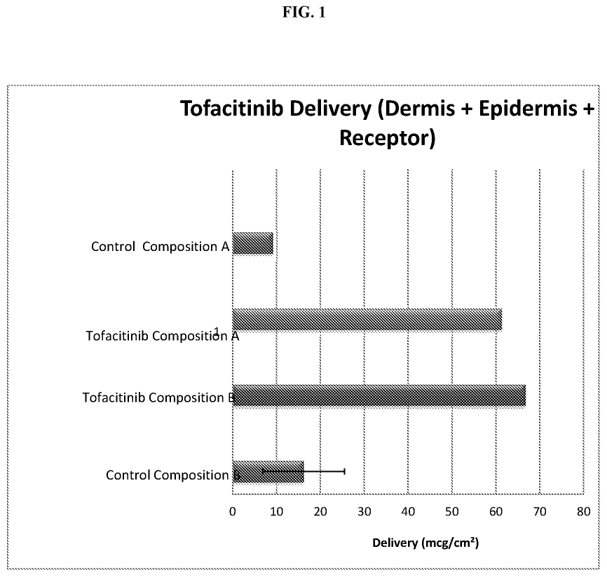

Cumulative skin flux of tofacitinib was shown in FIG. 1. The data demonstrated that both compositions containing ionic liquid according to non-limiting embodiments of the present disclosure resulted in a significantly increased permeation of tofacitinib compared to the two control compositions that includes oleyl alcohol and Transcutol® as penetration enhancers.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of delivering tofacitinib to or through a skin, the method comprising administering to the skin a pharmaceutical composition comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.

2. The method of claim 1, wherein the pharmaceutical composition comprises about 0.01% to about 10% of tofacitinib or a pharmaceutically acceptable salt thereof.

3. The method of claim 1, wherein the pharmaceutical composition comprises about 0.1% to about 8% of tofacitinib or a pharmaceutically acceptable salt thereof.

4. The method of claim 1, wherein the pharmaceutical composition comprises about 0.1% to about 5% of tofacitinib or a pharmaceutically acceptable salt thereof.

5. The method of any one of claim 1, wherein the pharmaceutical composition allows therapeutically effective amount of tofacitinib to reach at least one of an epidermis layer and a dermis layer.

6. The method of claim 5 wherein the amount of tofacitinib in the at least one of an epidermis layer and a dermis layer is sufficient to inhibit activity of at least one of JAK-1, JAK-2, TYK-2, and JAK-3 in the at least one of an epidermis layer and a dermis layer.

7. The method of claim 5, wherein the total amount of tofacitinib reaching at least one of an epidermis layer and a dermis layer provided by the pharmaceutical composition is at least 1.5 times the total amount of tofacitinib reaching at least one of an epidermis layer and a dermis layer provided by a control composition replacing the ionic liquid with same amount of 2-(2-ethoxyethoxy)ethanol.

8. The method of claim 5, wherein the total amount of tofacitinib reaching at least one of an epidermis layer and a dermis layer provided by the pharmaceutical composition is at least 2 times the total amount of tofacitinib reaching at least one of an epidermis layer and a dermis layer provided by a control composition comprising a PEG-based ointment and 2% of oleyl alcohol.

9. The method of any one of claim 5, wherein the pharmaceutical composition allows therapeutically effective amount of tofacitinib to reach at least an epidermis layer.

10. The method of any one of claim 5, wherein the pharmaceutical composition allows therapeutically effective amount of tofacitinib to reach at least a dermis layer.

11. The method of any one of claim 1, wherein the pharmaceutical composition allows therapeutically effective amount of tofacitinib to reach beyond the dermis layer.

12. The method of claim 1, wherein the fatty acid is selected from the group consisting oleic acid, geranic acid, hexanoic acid, and malonic acid.

13. The method of claim 1, wherein the fatty acid is geranic acid.

14. The method of claim 1, wherein the ionic liquid is a deep eutectic solvent (DES).

15. The method of claim 1, wherein the ionic liquid comprises the choline cation and fatty acid anion in a molar ratio in a range of 1:1 to 1:4 of choline cation to fatty acid anion.

16. The method of claim 1, wherein the ionic liquid comprises the choline cation and fatty acid anion in a molar ratio of 1:2 of choline cation to fatty acid anion.

17. The method of claim 1, wherein the pharmaceutical composition further comprises a pharmaceutically acceptable carrier.

18. The method of claim 1, wherein the pharmaceutically acceptable carrier is an aqueous carrier.

19. The method of claim 1, wherein the pharmaceutical composition further comprises a penetration enhancer selected from 2-(2-ethoxyethoxy)ethanol or oleyl alcohol.

20. A topical composition, comprising tofacitinib or a pharmaceutically acceptable salt thereof, and an ionic liquid in an amount sufficient to allow a therapeutically effective amount of tofacitinib to reach a target depth within or beyond the skin, wherein the ionic liquid comprises a choline cation and a fatty acid anion.
